(19) United States
(12) Patent Application Publication
(10) Pub. No.: US 2025/0090116 A1
(43) Pub. Date: Mar. 20, 2025

(54) APPARATUS AND METHOD FOR IN VIVO BREAST TISSUE IMAGING USING CODED APERTURE XRAY SCATTER TOMOGRAPHY

(71) Applicant: CALIDAR, INC., Durham, NC (US)

(72) Inventors: Joshua Howard CARPENTER, Raleigh, NC (US); Stefan Matthias STRYKER, Durham, NC (US)

(21) Appl. No.: 18/727,521

(22) PCT Filed: Jan. 9, 2023
(86) PCT No.: PCT/US2023/060302

§ 371 Date: Jul. 9, 2024

Related U.S. Application Data
(60) Provisional application No. 63/297,793, filed on Jan. 9, 2022.

(51) Int. Cl. A61B 6/50; A61B 6/00; A61B 6/04; A61B 6/06; A61B 6/42; G06T 7/00; G06T 7/11; G06T 11/00; G06V 10/764; G16H 40/63

(52) U.S. Cl. CPC A61B 6/502; A61B 6/0414; A61B 6/06; A61B 6/4266; A61B 6/4291; A61B 6/5217; A61B 6/54; G06T 7/0012; G06T 7/11; G06T 11/005; G06T 11/006; G06T 11/008; G06V 10/765; G16H 40/63; G06T 2207/10081; G06T 2207/30068; G06T 2210/41; G06T 2211/441; G06T 2211/448; G06T 2211/452; G06V 2201/031

(57) ABSTRACT

A system and method for acquiring in vivo tomographic X-ray scatter data for tissue discrimination of breast tissue. The system includes a coded aperture for spatially encoding X-ray scatter originating from within a patient's breast. Detectors record the modulated scatter signal, which is used to reconstruct a spatially resolved estimate of the X-ray scatter spectra which can then be used to generate spatially resolved tissue type estimates for the user.

### CROSS-REFERENCE TO RELATED APPLICATIONS

This application claims priority to U.S. Provisional Patent Application No. 63/297,793, entitled “An apparatus and method for in vivo breast tissue imaging used coded aperture X-ray scatter tomography,” which was filed on Jan. 9, 2022, the entire contents of which is incorporated by reference herein in its entirety.

### BACKGROUND

Existing mammography systems image breast tissue in a transmission mode by measuring the attenuation of an X-ray source transmitted directly through the breast relative to the initial intensity of the X-ray source in one or more energy ranges. Mammograms from existing digital systems are either projections of a single view of the breast tissue from the X-ray source directly onto a 2D X-ray detector pixel array or, in a 3D mammography system, a 3D tomographic X-ray transmission image is computed from several projection views. In existing mammography systems without energy discrimination, projection images consist of grayscale values that are effectively controlled by tissue density. Tissue density can be similar enough between cancerous and benign tissue that it is difficult or impossible to differentiate with density alone. In systems with some energy discrimination, which can be achieved either through multi-energy channel detectors or multiple measurements with different source operating parameters or filtration, images composed of more than a single value per pixel can be computed, providing some additional contrast to features in the mammogram. Mammograms from these existing systems are inspected by a clinician for regions that they deem could potentially be malignant tissue. Whether they are true grayscale or multi-energy enhanced transmission-based images, mammograms from existing mammography systems provide limited information upon which the clinician must make a decision as to whether a region of tissue is cancerous, such that 70-80% of mammograms that are flagged for biopsy in the U.S. ultimately prove to be benign upon inspection of the biopsied tissue by a pathologist.

Approximately 30% of new female cancers are breast cancer, and there is an average risk of about 13% that a woman will develop breast cancer during their lifetime. According to the American Cancer Society, when breast cancer is detected early and in a localized stage then a woman has a 99% 5-year relative survival rate. Therefore, there is a need for a mammogram system that accurately detects breast cancer.

### FIELD OF THE INVENTION

The present invention is generally directed to medical imaging, more specifically, the present invention is directed to mammography and X-ray scatter tomography for creating spatially resolved volumetric X-ray scatter spectral reconstructions.

### DESCRIPTION OF RELATED ART

To date, the discriminatory power of X-ray scatter measurements for breast tissue has only been demonstrated ex vivo in relatively controlled sample and measurement scenarios, but the recent development of X-ray scattering methodologies that employ coded apertures allow for spatially resolved volumetric X-ray scatter spectral reconstructions to be implemented in a sample and measurement scenario similar to those of a typical mammography system. An in vivo coded aperture-based X-ray scatter imaging system and methodology offers additional spatially resolved estimates of the likelihood a region of tissue is cancerous to a clinician, based on information that would never be accessible in a transmission-only mammography system.

### BRIEF SUMMARY OF THE INVENTION

The subject matter described herein includes a system and method for coded aperture-based X-ray scatter tomography of breast tissue in vivo. With this system and method, spatially resolved volumetric X-ray scatter spectral reconstructions of breast tissue in vivo are used to provide spatially resolved estimates of tissue type. Specifically, these estimates may include the likelihood that regions of breast tissue are cancerous based on comparisons to reference X-ray spectra of benign and cancerous tissue types. Given that 70-80% of mammograms flagged for biopsy prove negative, such estimates provide valuable additional information to aid clinicians in making biopsy decisions. No known prior art provides spatially resolved volumetric X-ray scatter spectral reconstructions or derived tissue type likelihood estimates calculated from X-ray scatter spectral data for breast tissue in vivo.

In contrast to X-ray transmission measurements of tissue, which can only provide the same number of features per pixel as number of effective energy channels in the measurement, spatially resolved volumetric X-ray scatter spectral reconstructions provide as many features as allowed by the energy resolution, the geometry of the measurement (particularly the size and pixel pitch of the detector when acquiring in a mostly angle-dispersive mode), and the accuracy of the model used for reconstruction. These additional features are also largely orthogonal from those provided by X-ray transmission measurements, i.e., they provide distinct information from any transmission X-ray measurement modality. More importantly for informing a clinician, transmission X-ray measurements are inherently limited by the similar and potentially overlapping density values of malignant and benign tissue, while X-ray scatter measurements of tissue have been shown to distinguish between cancerous and benign tissue ex vivo.

The mammography system described herein comprises a movable X-ray source for generating a primary X-ray beam for irradiating a breast of a patient. The X-ray source is adjustable based on at least one operating parameter that includes exposure time, current, voltage, or filtration. The mammography system further comprises a collimator between the X-ray source and the breast of the patient to shape the primary X-ray beam. The collimator includes an opening that is configurable in at least one dimension. The mammography system further includes a plurality of movable breast plates to position the breast of the patient in a path of the primary X-ray beam. The mammography system further comprises an X-ray detector array comprising a plurality of movable X-ray detecting elements. The X-ray detector array is configurable to position at least one X-ray detecting element distally from the X-ray source at a first measurement location in the path of the primary X-ray beam to measure transmitted X-ray radiation from the primary X-ray beam. The X-ray detector array is further configurable to position at least one X-ray detecting element distally from the X-ray source at a second measurement location outside the path of the primary X-ray beam past the breast of the patient to measure scattered X-ray radiation from the primary X-ray beam. The mammography system further comprises a coded aperture positioned distally from the X-ray source between the breast of the patient and the X-ray detector array, wherein the coded aperture is configured to modulate scattered X-ray radiation from the breast of the patient detected by the X-ray detector array. The mammography system is configurable to perform an X-ray scatter measurement and an X-ray transmission measurement. When performing the X-ray scatter measurement, the X-ray detector array is configured such that at least one X-ray detecting element of the plurality of X-ray detecting elements is positioned out of the path of the primary X-ray beam to detect scattered X-ray radiation from the primary X-ray beam passing through the breast of the patient. When performing the X-ray transmission measurement, the X-ray detector array is configured such that at least one X-ray detecting element of the plurality of X-ray detecting elements is positioned to detect X-rays transmitted through the breast of the patient from the primary X-ray beam. The mammography system further comprises a control system comprising memory and a processor. The processor is configured for configuring the mammography system for an X-ray transmission measurement or an X-ray scatter measurement. Configuring the mammography system includes controlling at least one configuration parameter. The at least one configuration parameter includes a position of the X-ray source, a position of the X-ray detector array, a dimension of the opening of the collimator, at least one operating parameter of the X-ray source, and a position of the plurality of breast plates. The processor is further configured for receiving X-ray transmission data detected by the X-ray detector array. The processor is further configured for producing an X-ray radiodensity mammogram image based on the received X-ray transmission data. The processor is further configured for identifying a region of interest in the breast of the patient based on the X-ray radiodensity mammogram image. The processor is further configured for determining at least one scatter configuration parameter for the X-ray scatter measurement based on the identified region of interest in the breast of the patient. The processor is further configured for configuring the mammography system for the X-ray scatter measurement based on the at least one determined scatter configuration parameter. The processor is further configured for receiving X-ray scatter data detected by the X-ray detector array. The processor is further configured for estimating a spatially resolved X-ray scatter spectral reconstruction based on the received X-ray scatter data, the received X-ray transmission data, and the at least one scatter configuration parameter. The processor is further configured for determining a spatially resolved tissue property based on the received X-ray scatter data of the region of interest. The processor is further configured for producing a spatially resolved scatter mammogram image based on the received X-ray transmission data and the received X-ray scatter data. There are many potential embodiments of the mammography system, several of which are described further below.

The method of performing spatially resolved volumetric X-ray scatter tomography mammography described herein comprises performing an X-ray transmission measurement. The X-ray transmission measurement comprises transmitting a first primary X-ray beam from an X-ray source through a collimator having an opening configurable in at least one dimension to shape the first primary X-ray beam and through a breast of a patient positioned between a plurality of breast plates. The X-ray source is adjustable based on at least one operating parameter that includes exposure time, current, voltage, or filtration. The X-ray transmission measurement further comprises detecting X-ray radiation from the first primary X-ray beam transmitted directly through the breast of the patient using at least one X-ray detecting element of an X-ray detector array. The X-ray transmission measurement further comprises receiving X-ray transmission data via a control system including a processor and memory from the X-ray detector array. The method further comprises producing a radiodensity mammogram image of the breast of the patient based on the received X-ray transmission data. The method further comprises identifying a region of interest in the breast of the patient based on the radiodensity mammogram image. The method further comprises determining at least one scatter measurement configuration parameter for an X-ray scatter measurement of the identified region of interest in the breast of the patient. The scatter measurement configuration parameter includes a position of the X-ray source, a position of the plurality of the X-ray detecting elements, a dimension of the opening of the collimator, an X-ray source operating parameter, and a position of the plurality of breast plates. The method further comprises performing the X-ray scatter measurement of the region of interest. The X-ray scatter measurement comprises transmitting a second primary X-ray beam from the X-ray source through the collimator to shape the second primary X-ray beam and through the breast of the patient positioned with the plurality of breast plates. The X-ray scatter measurement further comprises modulating scattered X-ray radiation from the second primary X-ray beam interacting with the breast of the patient using a coded aperture positioned between the breast of the patient and the plurality of X-ray detecting elements. The X-ray scatter measurement further comprises detecting the modulated scattered X-ray radiation using the X-ray detector array. The X-ray scatter measurement further comprises receiving X-ray scatter data representing detected scattered X-ray radiation from the X-ray detector array. The method further comprises calculating a spatially resolved X-ray scatter spectral reconstruction based on the received X-ray scatter data, the received X-ray transmission data, and the determined scatter measurement configuration parameter. The method further comprises determining at least one spatially resolved tissue property based on the received X-ray scatter data of the region of interest. The method further comprises producing a spatially resolved scatter mammogram image based on the received X-ray transmission data and the received X-ray scatter data. There are many potential embodiments of the method, several of which are described further below.

Scattered X-ray radiation from breast tissue contains tissue-specific information. More specifically, tissue X-ray diffraction (XRD) spectra reflect local molecular ordering. For example, fat, gland, and cancerous tissue have different molecular ordering which results in distinct XRD spectra.

There are many potential embodiments of the system, several of which are described further below. The system includes at least one X-ray source to irradiate breast tissue. The system also includes at least one collimation stage between the X-ray source and the breast tissue. The system further includes a coded aperture positioned between the breast tissue and the detector. The system further includes a processor which can use the detected X-ray scatter signal, which has been modulated by the coded aperture, to compute a spatially resolved estimate of the tissue types of the irradiated breast tissue. Lastly, the system may further include a component for representing the tissue type estimate data to the operator or clinician, e.g., a computer monitor.

Molecular ordering also impacts density. In existing transmission mammography systems, the local molecular ordering is generally collapsed down to a single radiodensity value. However, the mammography system described herein is designed to generate spatially resolved X-ray scatter spectra of the region of breast tissue on which the scatter measurement and reconstruction are performed. The X-ray scatter spectra provides more features and to increase the contrast for differentiation of tissue types relative to existing mammography systems and the spatially resolved nature of the reconstructed data allows this differential ability to be associated with specific, known locations in the breast of the patient, and therefore correlated with observable features in radiodensity mammogram images produced by transmission measurements. The irradiance and X-ray energy spectrum reaching the breast tissue can be controlled using filters, which can be done to minimize the dose the patient receives as well as to improve contrast and X-ray spectral resolution. The cross-sectional shape (e.g., pencil, fan, or cone beam), divergence, and spatial extent of the X-ray beam reaching the breast tissue can be controlled with collimators between the X-ray source and the breast tissue. Second, the scattered X-rays are measured with an X-ray detector. A coded aperture, i.e., a patterned aperture with a known open fraction and pattern, is located between the breast tissue and the X-ray detector to selectively and purposefully attenuate the scattered X-rays based on the known code pattern and measurement geometry. Third, a spatially resolved estimate of the tissue types of the irradiated breast tissue is computed from the detected coded aperture modulated X-ray scatter signal using the processor. Finally, the estimates of the tissue types are presented to the operator or clinician using the display component. For example, and not limitation, the mammography system described herein is designed to generate a color map, overlaid onto an X-ray transmission image, corresponding to estimated tissue types and/or a likelihood of cancer. Additionally, the mammography system may flag a region of the transmission image that includes a value for the percentage of the likelihood of cancer. Advantageously, the mammography system is further operable to measure and display the X-ray scatter data of a subregion of the transmission data (e.g., scatter spot-checking during transmission magnification mode).

Spatially resolved volumetric X-ray spectral data can be reconstructed from an irradiated volume using a coded aperture to modulate the scatter, a 2D pixelated detector to measure the modulated scatter signal, a forward matrix model of the physics and the geometry of the measurement, and a processor to iteratively estimate, by commonly known algorithms (e.g., maximum likelihood estimation), the spatially resolved volumetric X-ray spectral data from the forward matrix model. This general procedure is used in the methods described herein. Specifically, this includes estimation of momentum transfer spectra on a pixel or voxel basis, which can be compared to reference momentum transfer spectra, such as those of cancerous tissue or benign adipose or gland tissue, or utilized in machine learning classification methods.

The specific embodiments discussed below include direct transmission measurements and are generally descriptive of embodiments of the system that have similar geometries, components, and form factors to existing mammography systems.

### BRIEF DESCRIPTION OF THE SEVERAL VIEWS OF THE DRAWINGS

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1 depicts an exemplary schematic of the general components and layout for embodiments of the spatially resolved volumetric X-ray scatter tomography mammography system described herein.

FIG. 2 depicts an exemplary process flow chart for performing embodiments of the spatially resolved volumetric X-ray scatter tomography mammography described herein.

FIG. 3 depicts a schematic of measured modulated X-ray scatter data, post-acquisition reconstruction calculation and presentation of a scatter mammogram with a tissue property, in this example, a suspicious target's match with cancer, with an illustration of the reconstructed scatter spectral dimension used to calculate the tissue property according to one embodiment described herein.

FIG. 4A depicts the collimator in an open position for changing field of view or beam shape, in this example, to create a cone beam, according to one embodiment described herein.

FIG. 4B depicts a multi-stage collimator producing a pencil or fan beam, according to one embodiment described herein.

FIG. 5A depicts a schematic example of a coded aperture for a pencil beam according to one embodiment described herein.

FIG. 5B depicts a schematic example of a coded aperture for a fan beam according to one embodiment described herein.

FIG. 6A depicts a schematic example of a mammography system with a beam block separate from and behind a coded aperture according to one embodiment described herein.

FIG. 6B depicts a schematic example of a mammography system with a beam block separate from and in front of a coded aperture according to one embodiment described herein.

FIG. 6C illustrates an X-ray system with a beam block built into a coded aperture according to one embodiment described herein.

FIG. 7A depicts a mammography system in a magnification mode performing a transmission measurement and identifying a region of interest in the transmission mammogram of the breast according to one embodiment described herein.

FIG. 7B depicts a mammography system in a magnification mode performing a scatter measurement of an identified region of interest and generating a scatter mammogram with an indication of a spatially resolved estimate of a tissue property in this example whether or not the region of interest contains cancerous tissue, according to one embodiment described herein.

FIG. 8A depicts a mammography system with a rotating coded aperture in a folded position according to one embodiment described herein.

FIG. 8B depicts a mammography system with a rotating coded aperture in an unfolded position according to one embodiment described herein.

FIG. 9A depicts a schematic embodiment of a mammography system including a movable transmission detector in an active position according to one embodiment described herein.

FIG. 9B depicts a schematic embodiment of a mammography system including a movable transmission detector in an inactive position according to one embodiment described herein.

FIG. 10A depicts an X-ray detector array according to one embodiment described herein.

FIG. 10B depicts an X-ray detector array comprising a plurality of X-ray detectors in a linear orientation according to one embodiment described herein.

FIG. 10C depicts an X-ray detector array comprising a plurality of X-ray detectors in a curved orientation according to one embodiment described herein.

FIG. 10D depicts an X-ray detector array comprising a plurality of X-ray detectors in a spaced-out orientation according to one embodiment described herein.

FIG. 11 depicts a schematic diagram of a mammography system comprising a second X-ray source used for performing X-ray scatter measurements, which can be oriented for X-ray scatter measurement during a magnified and non-magnified mammogram exam, according to one embodiment described herein.

FIG. 12A depicts a schematic diagram of a mammography system operating in transmission mode including an X-ray source for X-ray transmission measurements and X-ray scatter measurements using separate X-ray detectors according to one embodiment described herein.

FIG. 12B depicts a schematic diagram of a mammography system operating in scatter mode including an X-ray source for X-ray transmission measurements and X-ray scatter measurements using separate X-ray detectors according to one embodiment described herein.

FIG. 13 depicts a schematic diagram of a mammography system with a detachable coded aperture according to one embodiment described herein.

FIG. 14 depicts a schematic diagram of a mammography system with a detachable coded aperture that is affixed to a magnification spacer platform according to one embodiment described herein.

FIG. 15 depicts a schematic diagram of a spatially resolved scatter mammogram image generated from X-ray transmission data and X-ray scatter data with a region of interest for which scatter data is measured and reconstructed, with illustrations of reconstructed X-ray scatter spectra of the indicated voxels corresponding to different tissue types, according to one embodiment described herein.

FIG. 16 depicts a schematic of a spatially resolved scatter mammogram image generated from X-ray transmission data and X-ray scatter data with a region of interest for which scatter data is measured and reconstructed which is colorized based on the spatially resolved scatter reconstruction, illustrated by the hatching scale indicated, according to one embodiment described herein.

### DETAILED DESCRIPTION

For the purposes of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise,” “consist of,” or “consist essentially of,” the steps, elements, and/or reagents described in the claims.

It is further noted that the claims may be drafted to exclude any optional element. As such, this statement is intended to serve as antecedent basis for use of such exclusive terminology as “solely,” “only,” and the like in connection with the recitation of claim elements, or the use of a “negative” limitation.

In the descriptions herein, the measured scatter signal and reconstructed spatially resolved scatter spectra are referred to as pertaining to the “X-ray scatter,” though the X-ray scatter field will in general be comprised of both Rayleigh and Compton scatter. The use of the measured scatter signal and reconstructed spatially resolved scatter spectra and similar terminology, particularly the use of the term “diffraction,” is not intended to limit the present invention to pertaining to scatter arising from one physical process and not another.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

The subject matter described herein is generally directed to mammography, and more specifically to mammography systems that use coded apertures to modulate the scattered X-ray radiation from the irradiated breast of a patient. More specifically, the subject matter described herein is directed to mammography systems that measure the modulated scattered X-ray radiation from the breast for estimating a spatially resolved volumetric X-ray scatter tomography spectral reconstruction of the breast. Spatially resolved volumetric X-ray scatter tomography is defined herein as the measurement of X-ray scatter spectra throughout a tomographic image, such that each pixel or voxel contains an additional dimension of data which is the spatially resolved scatter measurement. This tomographic image is obtained by conducting X-ray scatter measurements. The X-ray scatter measurements include detecting X-rays scattering from tissues and processing the X-ray scatter measurement data to localize scattered X-rays angle and origin from within the tissue. Such mammography systems are designed to measure spatially resolved structural differences in a patient's body at a cellular level as reflected in the spatially resolved volumetric X-ray scatter spectral reconstruction.

Existing X-ray mammography systems comprise X-ray sources to produce X-rays and an X-ray detector to capture the X-rays after passing through the patient's tissue. In this manner, existing X-ray mammography systems operate in a transmission mode and perform X-ray transmission measurements which are used to generate transmission, or X-ray radiodensity, mammogram images. The X-ray radiodensity intensity scale is based on the amount of X-rays transmitted through the tissue. The measurement of the X-rays transmitted through the tissue represents the X-ray transmission measurement. X-ray radiodensity mammogram images can be two dimensional or three dimensional, with three dimensional mammogram images being tomographically reconstructed from X-ray transmission measurements from multiple perspectives of the tissue. Clinicians evaluate X-ray radiodensity mammograms based on the shape and radiodensity of features observed in the tissue. Adipose tissue is less dense than other types of breast tissue, whereas cancerous tissue can have similar density to healthy gland tissue or benign masses. Calcifications in the tissue are more dense than adipose, glandular, and cancerous tissues, as well as fibroadenoma, but are not a definitive indication of the presence of cancerous tissue. Therefore, in general, cancerous tissue cannot be unambiguously distinguished from healthy tissue in X-ray radiodensity mammograms.

Beam divergence results in X-ray beams becoming larger in area and less intense the further the distance from the X-ray source. This further causes some magnification of an object being radiographed. The basic principle of transmission X-ray imaging is that X-rays travel in straight lines. However, when X-ray scattering events occur in the patient, the resulting scattered X-rays are not aligned with the trajectory of the original primary X-ray beam. In existing transmission mammography systems, scattered radiation detected by the X-ray detector can be a significant cause of image degradation. Scattered radiation generates undesired image intensity that is not indicative of the radiodensity of the tissue along the straight line path from the source in the region of the image in which it is detected, which can significantly reduce contrast. Contrast can be increased in digital radiodensity images by windowing, leveling, or other adjustment schemes, so for digital radiographic images, scatter acts chiefly as a source of noise, degrading the signal-to-noise ratio (SNR).

Therefore, in transmission mammography, it is typically important to control the amount of scatter radiation reaching the X-ray detector to create a high-quality image. Scatter radiation causes unwanted exposure to an image. To address the undesired image intensity generated in transmission mammograms by scattered radiation, anti-scatter grids are used to absorb scatter radiation exiting a patient and reduce the amount of scatter reaching the image detector. An anti-scatter grid is a component placed near a detector that is designed to angularly reject X-rays not originating from the X-ray source. The anti-scatter grid blocks many of the scattered X-rays that, in a transmission measurement, are a source of noise or image degradation. In contrast, for volumetric X-ray diffraction imaging, these scattered X-rays are the desired source of signal that need to be measured. For these measurements, the anti-scatter grid is removed from the system. The coded aperture component is positioned between the breast and detector, but not directly against the detector like the anti-scatter grid. The coded aperture absorbs some of the scattered X-rays while letting others pass through to the detector. The absorption of some of the scattered X-rays generates unique shadows within the measured scatter data that an algorithm (e.g., maximum likelihood estimation) benefits from, enabling the computation of scattered X-ray origins and scattered angles that generate the X-ray scatter spectra of the volumetric X-ray diffraction image.

In addition to beam-modifying and/or restricting devices, two major factors affect the amount and energy of scatter radiation exiting the patient's tissue: kilovoltage peak (kVp) and the volume of tissue irradiated. The volume of tissue depends on the thickness of a region of interest, the sub-region within a patient that an operator is interested in examining, and the X-ray beam field size. Increasing the volume of tissue irradiated results in increased scatter production. In addition, using a higher kVp increases X-ray transmission and reduces its overall absorption (photoelectric interactions); however, higher kVp increases the percentage of photon interactions (e.g., Compton interactions, Thomson scattering) and the energy of scatter radiation exiting the patient. Using higher kVp or increasing the volume of tissue irradiated results in increased scatter radiation reaching the image detector.

While performing mammography, one goal is to limit the beam field size to an area of interest. In order to limit the field size, the X-ray beam is restricted to limit patient exposure and reduce the amount of scatter radiation produced. For example, an unrestricted beam projects beyond the boundaries of the image detector and increases patient exposure. Increasing the collimation decreases the volume of tissue irradiated, the amount of scatter radiation produced, the number of photons that strike the patient, and the number of X-ray photons reaching the image detector to produce the latent image. Therefore, in order to improve a radiodensity mammography image by limiting scatter radiation, a mammography system should include controllable beam-modifying and/or restricting devices and/or components. For X-ray scatter measurements with the present invention, controlling the region of interest of irradiated tissue via collimation allows for measuring less scatter from non-suspicious regions of tissue so, while the total strength of the full scattered radiation field is decreased, there is less multiplexing of scatter from non-suspicious regions of tissue in the total measured scatter signal, allowing for measuring more useful scatter signal with less total radiation dose to the patient.

To address the aforementioned problems, in one embodiment, the mammography system described herein includes an X-ray source (x-ray generator), an X-ray tube, a collimator, at least one compression plate, anti-scatter grid, a coded aperture, and an image detector. The X-ray source is designed for delivering, modulating, and regulating electrical energy required by the X-ray tube. The X-ray tube is designed to emit, accelerate, and decelerate electrons to produce X-radiation. The voltage applied between the electrodes (cathode and anode) accelerates the electrons. The anode material determines the characteristics of the radiation. The X-ray source and X-ray tube affect the radiation yield, exposure time, and image quality. The collimator controls the shape of the X-ray beam and subsequent illuminated tissue volume. The compression plate is manually and/or automatically adjustable to alter the thickness and pressure on a region of interest of a patient. Advantageously, for transmission measurements, adequate compression results in less scattering of radiation, improved contrast due to less beam hardening, reduced radiation dose, prevents motion blurring, better image geometry, and better tissue separation.

In one embodiment, the mammography system includes an auxiliary filter placed in the X-ray beam path to modify the photon spectrum of the primary X-ray beam and to optimize radiation quality. For example, and not limitation, the filter material includes, but is not limited to, molybdenum, rhodium, copper, and aluminum. In another embodiment, the collimator is designed to decrease a patient dose, scatter radiation, and exposure to image receptor while increasing radiographic contrast.

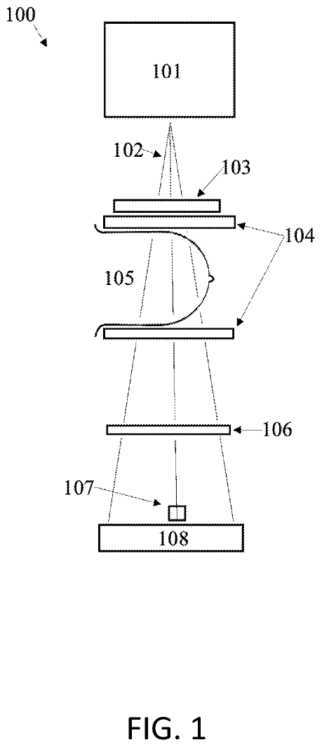

A general depiction of such an embodiment of the mammography system is shown in FIG. 1. FIG. 1 depicts a schematic of the general components and layout for X-ray transmission measurements and X-ray scatter measurements of in vivo breast tissue. Mammography system 100 comprises a movable X-ray source 101 for generating an X-ray beam 102 for irradiating a breast 105 of a patient. The X-ray source 101 may be adjustable based on at least one operating parameter, which may include exposure time, current, voltage, or filtration. Mammography system 100 further comprises a collimator 103 with an opening that is configurable in at least one dimension positioned between the X-ray source and the breast of the patient to control the primary X-ray beam 102 shape and illuminated tissue volume. Mammography system 100 further comprises a plurality of breast plates 104 to position a patient breast 105. Mammography system 100 further comprises a coded aperture 106. Coded aperture 106 is positioned between the breast 105 of the patient and the X-ray detector array 108. In one embodiment, coded aperture 106 may be integrated into a breast plate 104. Coded aperture 106 is configured to modulate scattered X-ray radiation from the patient breast 105 detected by the X-ray detector array 108. X-ray detector array 108 comprises a plurality of X-ray detecting elements. X-ray detector array 108 is movable in at least two dimensions and is configurable for positioning at a measurement location to measure X-ray radiation from the X-ray beam 102. Mammography system 100 may further include an X-ray beam block 107. Beam block 107 may be movable. Beam block 107 may be positioned between the patient breast 105 and the X-ray detector array 108 in the path of the X-ray beam 102. In one embodiment, beam block 107 may be integrated into coded aperture 106. Note that movable and moving is defined here not only as components moving in physical space, but also could be achieved by moving a patient relative to the components (e.g., motion on a platform or bed that could move a patient in 3D space while keeping the system stationary).

Mammography system 100 separately performs X-ray transmission measurements and X-ray scatter measurements and uses different configurations for the X-ray transmission measurements versus the X-ray scatter measurements. More specifically, mammography system 100 performs an initial X-ray transmission measurement or set of X-ray transmission measurements with accompanying data processing to produce two-dimensional (2D) or three-dimensional (3D) transmission mammograms that are typical of existing digital mammography methods, followed by a subsequent X-ray scatter measurement or set of X-ray scatter measurements and associated data processing to calculate spatially resolved X-ray scatter spectral reconstructions and the associated estimates of tissue type for the illuminated tissue volume in the X-ray scatter measurement, as explained in more detail below. Spatially resolved X-ray scatter spectral reconstructions are defined here as the multi-dimensional data or image that contains X-ray scatter spectra within each spatial location, as reconstructed (or computed) by a reconstruction algorithm (e.g., maximum likelihood estimation). Advantageously, mammography system 100 identifies a region of interest of the transmission mammogram to perform X-ray scatter measurements and tissue type analysis, which minimizes a radiation dose to the patient, improves speed of the total measurement and analysis process, and facilitates adoption of the methods by clinicians, relative to a method that performs scatter measurements of the entire breast. Alternatively, or additionally, mammography system 100 may receive a selection of the region of interest via a user interface. Transmission mammogram data can also be used jointly with reconstructed estimates of pixel or voxel scatter spectra to classify tissue.

The mammography system described herein is designed for scattered X-rays to pass through the coded aperture such that the intensity of the scattered X-rays is modulated based on the aperture pattern. This results in different magnifications and projections of shadows in the scatter data. In addition to the spatially dependent intensity modulations, different tissue types have different scatter signatures. The mammography system may simultaneously measure all irradiated tissue points. The raw modulated scatter data is summed from all irradiated tissue points. The measured scatter data may be processed (e.g., background subtraction or Fourier filtration). The mammography system reconstructs X-ray scatter spectra of the object space, where there are the number of chosen object dimensions plus an additional dimension for the scatter data. This can include a reconstruction with three spatial dimensions and a fourth scatter dimension. In such an example, the reconstruction would consist of 4D pixels, referred to in some contexts as toxels. As an example, and not limitation, using a system of linear equations, the mammography system reconstructs the X-ray spectral data of the object space by vectorizing the entire pixelated intensity of scatter data, then vectorizing the entire reconstructed spatially resolved object space spectral map. Next, the mammography system creates a forward matrix that represents the forward projection of the scatter X-rays from an object vector to a measurement vector. As an example, the detected scatter intensity vector equals the forward matrix multiplied by the XRD map vector. The forward matrix may include a spatial model of the coded aperture with attenuation properties and locations relative to each point in object space and each point in the detector space. The mammography system may further include at least one algorithm to estimate the object space using the measured detector space and a model of the XRD map system. The coded aperture enables differentiation in the X-ray signal based on location in the breast tissue to accurately estimate XRD spectra in each voxel and generate a toxel map.

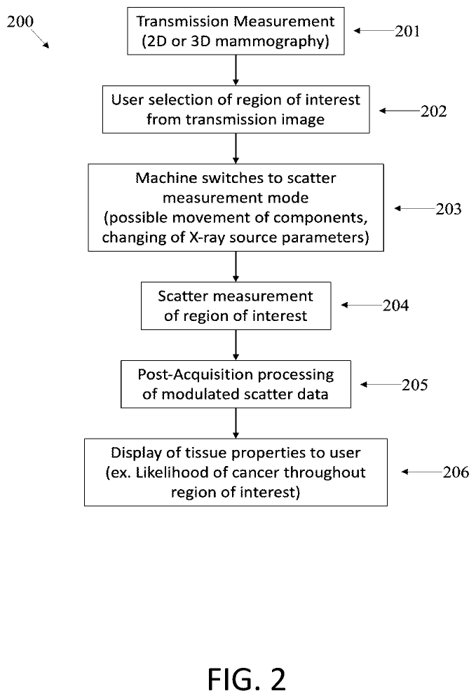

FIG. 2 depicts an exemplary process flow chart for embodiments of the method of performing spatially resolved volumetric X-ray scatter tomography mammography described herein. In method 200, a standard mammogram (i.e., an X-ray transmission measurement) is performed first 201, producing a two-dimensional (2D) or three-dimensional (3D) radiodensity mammogram image of the breast. Next, in method 200, a region of interest is selected 202 by a user via a user interface. Next, in method 200, a control system configures the mammography system 203 an X-ray scatter measurement of the identified region of interest 204. To configure the mammography system 203 the control system calculates how to move components or change measurement parameters in order to modulate scattered X-ray radiation from the specified region of interest with the coded aperture and measure the modulated scatter with the X-ray detector array. In some embodiments this calculation involves optimizing the configuration to minimize radiation dose to the patient or improve a quality, such as signal to noise, of the measured X-ray scatter data to be measured. The control system then enacts the configuration by moving components or changing measurement parameters and performs the X-ray scatter measurement of the identified region of interest 204. For example, and not limitation, X-ray scatter measurements include using the coded aperture to modulate the scatter, so the mammography system is operable to move the other components into a correct relative position. For example, the collimator may modify the beam size and shape and the source could be moved to target a region of interest. After an X-ray scatter measurement 204, reconstruction and classification algorithms operate on the collected scatter data 205 to provide estimated tissue properties, an example being the likelihood of malignant tissue 206, and produce a spatially resolved scatter mammogram image 206. Note that a spatially resolved scatter mammogram image here can mean a transmission mammogram image (e.g., 2D, 3D) that includes additional data provided from a scatter measurement. This could be as complex as showing data from the scatter measurement throughout the entire breast, in a region of interest, or showing a summary metric (e.g., binary yes/no cancer, max probability of cancer in entire breast) that is represented in a non-spatial manner.

For example, and not limitation, in one embodiment, the X-ray source is configured to generate a pencil beam to perform an X-ray scatter measurement and acquire data for a region of interest flagged by a user and/or the software of the mammography system. The mammography system determines that the X-ray source may be moved to change the perspective of the breast, provide a shorter beam through the tissue, vary types of tissue the beam passes through (e.g., to minimize the amount of gland tissue in the beam path), and generally to target the region of interest. Decreasing the amount of tissue that the X-ray beam passes through will result in less attenuation of the scatter X-ray signal and therefore a lower amount of radiation dosage needed for the same signal to noise quality of the measured scatter data. Additionally, the collimator is adjustable to move the pencil beam from the X-ray source through the region of interest to minimize the beam path. The mammography system may move the beam block so the direct X-ray beam is blocked from reaching the detector. The detector and/or coded aperture may be moved to optimize the X-ray scatter measurements (e.g., capture more signal or a more relevant region of the scatter radiation field in a shorter amount of time). The mammography system may further adjust X-ray exposure time, an amount of current, and/or voltage to optimize dosage and the measurement signal-to-noise ratio.

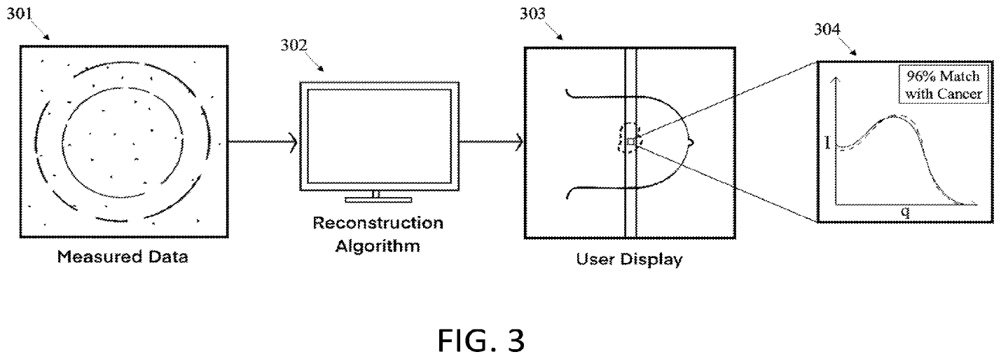

FIG. 3 depicts a schematic of the post-acquisition process conducted on measured scatter data to present a system operator with tissue information, in this example, a suspicious target's match with cancer. Demonstrating the conversion of modulated scatter data into information for an operator, FIG. 3 presents a schematic where the “Measured Data” panel 301 gives example scatter data with spatially varying intensity modulations due to the coded aperture. Reconstruction algorithms (e.g., maximum likelihood estimation) can be used along with a forward model 302 of the measurement scenario to demultiplex the measured modulated scatter signal in which the spatial origin of the scatter signal and scatter spectra associated with each position are convolved, thereby, allowing for a reconstruction of the spatially resolved X-ray scatter spectral information. The “User Display” panel 303 shows an example of how the information may be presented to an operator, with the window 304 showing a schematic of measured and reference scatter signature and cancer comparison with an indicator of a score or match between the measured signature and the reference cancer signature. For example, and not limitation, the score or match between the reconstructed X-ray scatter spectrum in a voxel and a reference spectrum of a tissue type could be provided to the user by colorization of 2D or 3D transmission mammogram images, overlaying a text box with the match or score value onto the image, overlaying a text box onto the image with an indication that the match or score value is above a threshold, binary flagging of regions of interest as benign versus cancerous, and/or providing a continuum of values for each pixel or voxel informing a user on a region of interest (e.g., correlation with reference X-ray scatter spectra of cancerous tissue). For example, and not limitation, the mammography system may display a metric or score (e.g., estimated percentage of likelihood of cancer for a voxel and/or a region of voxels).

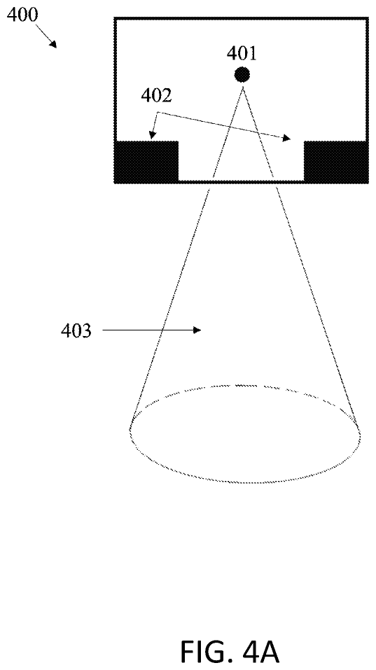

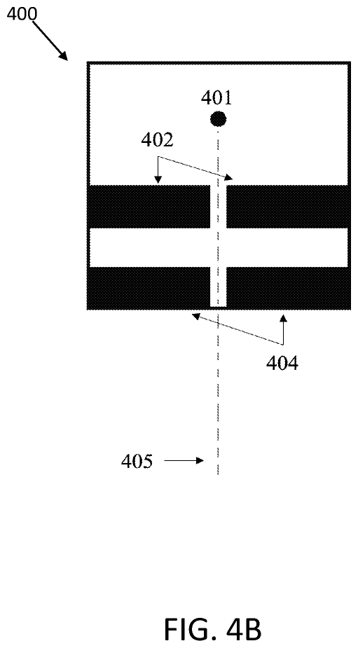

Some specific features of the components in these similar embodiments of the system are shown in FIGS. 4A, 4B, 5A, 5B, 6A, 6B, and 6C. FIGS. 4A and 4B depict how the collimation and subsequently the irradiated volume of tissue may be changed within the X-ray source head 400 by opening the collimation 402 or varying the number and location of collimators shaping the beam 404. FIG. 4A shows that X-ray focal point 401 produces a cone beam 403 due to the open collimation. FIG. 4B illustrates how the X-ray focal point produces a pencil or fan beam 405 due to the multi-stage collimation of 402 and 404. This ability to control the illuminated volume of tissue is common among the specific embodiments of the method discussed below. For example, and not limitation, the collimator may be used to switch to a pencil beam to target a suspicious region of interest. This reduces the illuminated volume of tissue relative to open collimation, which would decrease the radiation dose to the patient and simplify the X-ray scatter data processing and reconstruction by reducing the overall level of multiplexing in the X-ray scatter data.

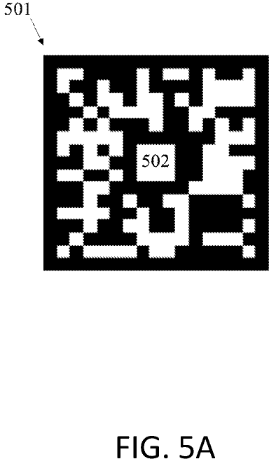

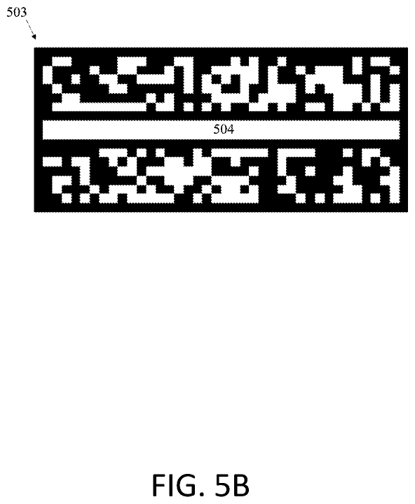

FIGS. 5A-5B show two example coded apertures that may be used in the embodiments described herein. The coded aperture 501 in FIG. 5A has a central opening 502 that allows a primary pencil beam to pass through without interaction. FIG. 5B shows a coded aperture 503 including a central slit 504 for a primary fan beam to pass through. This concept may be extended by having a larger central opening for a cone beam if used during the X-ray scatter measurement. The embodiments with openings in the aperture would be utilized if the beam block is positioned after a coded aperture, while these openings could instead be replaced with beam blocks built into the aperture in an alternative embodiment.

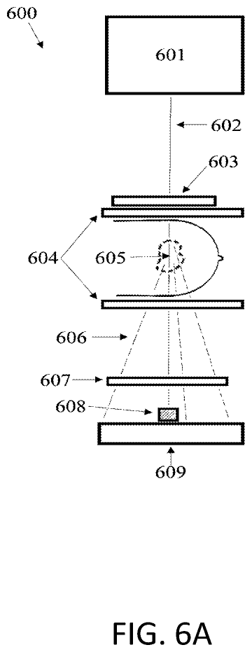

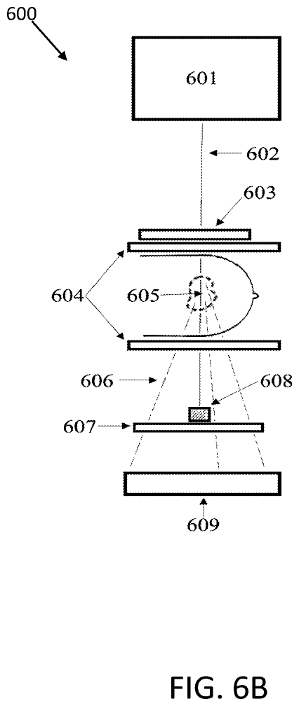

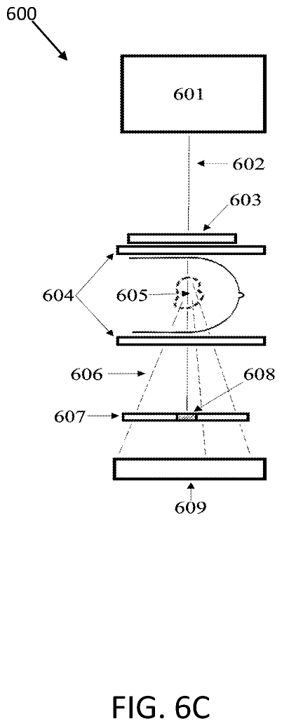

FIGS. 6A-6C show how beam blocks 608 may be implemented in the embodiments of the mammography system 600 described below. Shown in FIGS. 6A-6C is an X-ray source 601, primary X-ray beam 602, collimator 603, breast plates 604, tissue region of interest within patient breast 605, scattered X-rays 606, coded aperture 607, primary beam block 608, and detector 609. The utility of beam blocks for X-ray scatter measurements is known to those who are skilled in the art. The beam block may be separate from the coded aperture and may be located in front of or behind the coded aperture, relative to the tissue location, as shown in FIGS. 6A and 6B, or it may be built into the coded aperture, as shown in FIG. 6C. Such choices of beam block implementation would be made by considering tradeoffs between ease of integration of the beam block into the form factor of existing transmission mammography systems and quality of the measured scatter data (i.e., to reduce excess parasitic scatter signal from the coded aperture or other components) among other considerations. Embodiments of the mammography system with similar beam block configurations may also have the ability to translate or rotate any of these beam block configurations, especially in an embodiment where sources and collimators can be moved or varied to illuminate specific subregions of the breast tissue.

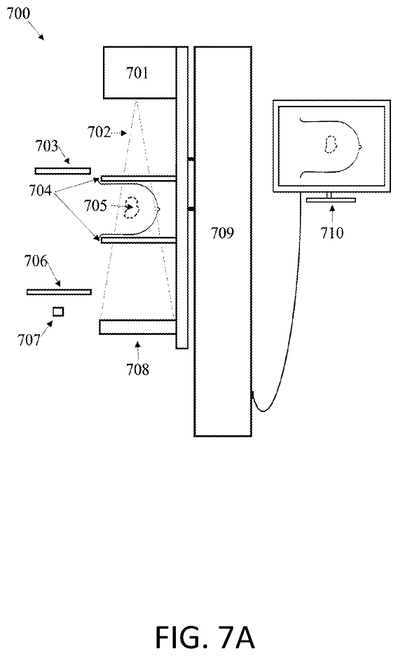

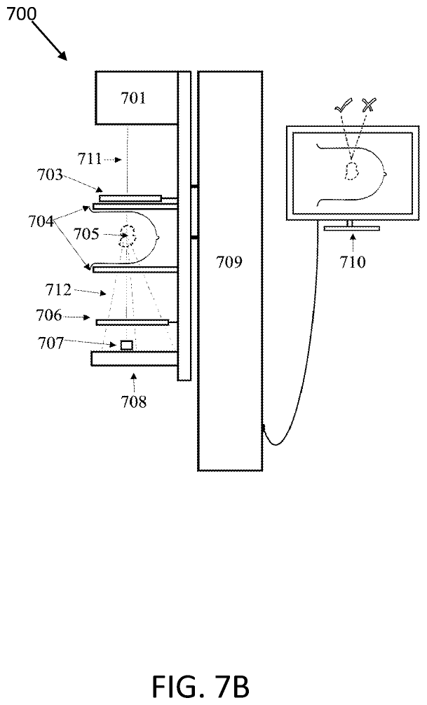

An embodiment of the mammography system that facilitates demonstration of the first transmission measurement and second scatter measurement of a region of interest, or switching between a transmission measurement and a scatter measurement is shown in FIGS. 7A and 7B. For the mammography system 700 shown in FIGS. 7A and 7B, there is an X-ray source 701, X-ray cone beam 702, collimator 703, breast plates 704, tissue region of interest within patient breast 705, coded aperture 706, primary beam block 707, detector 708, component mounting/rotation system 709, display for acquired results 710, X-ray beam for X-ray scatter measurements 711, and scattered X-rays 712. FIG. 7A shows an embodiment of the mammography system in a magnification mode, where the breast is positioned with the breast plates 704 at a distance further from the detector 708 than when measuring in a standard or non-magnified mode. In an embodiment of the system with an anti-scatter grid that covers the detector for use in a non-magnified transmission mode, the anti-scatter grid would retract. FIG. 7B shows collimation in the X-ray source head shaping the cone beam into a pencil or fan beam, with a collimator 703 moved into the beam path to decrease background scatter. The pencil or fan beam illuminates the suspicious region of the breast tissue 705. A movable coded aperture 706 is also configured in front of the detector to modulate the intensity of the scattered X-ray radiation reaching the detector and a movable beam block 707 is also configured into the path of the pencil or fan beam used for the X-ray scatter measurements. The tissue region of interest provided as feedback from the operator is converted by the control software into locations of collimator and beam block, to allow the X-ray scatter of only the suspicious region to be recorded in a second measurement. Alternatively, the tissue region of interest may be automatically flagged by the control software without feedback from an operator. The rest of the method for processing scatter data then proceeds as already described to reconstruct X-ray scatter spectra of the specified volume and provide the operator with a spatially resolved estimate of the likelihood of cancer in the specified subregion 710. This embodiment minimizes the dose received by the patient relative to doing both a full volume transmission measurement as well as a full volume diffraction measurement, but a full volume diffraction measurement with no operator specified sub-region may be conducted as well.

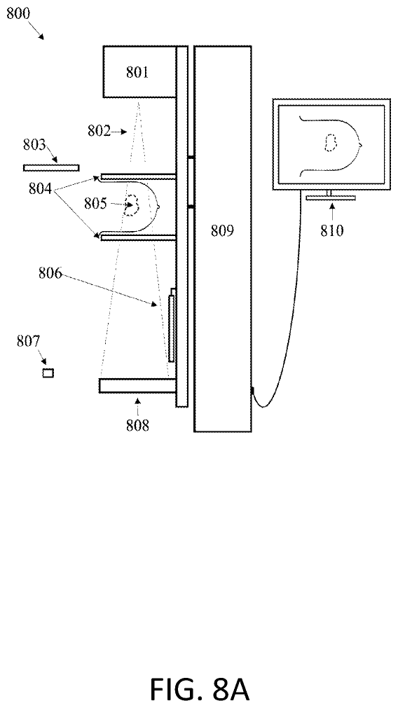

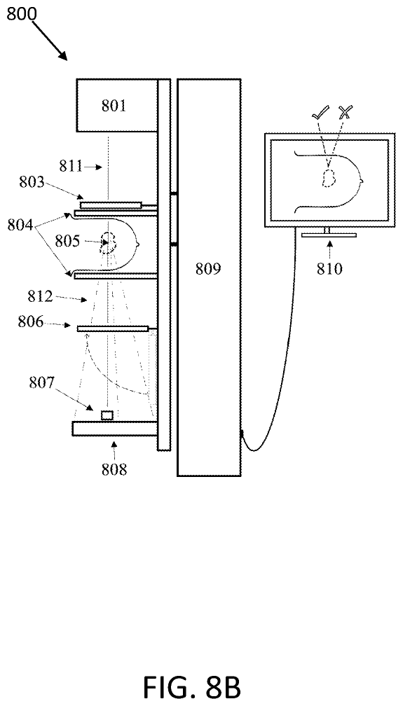

A similar embodiment of the mammography system to that shown in FIGS. 7A and 7B is shown in FIGS. 8A and 8B, with a coded aperture 806 that rotates into place for the X-ray scatter measurement as opposed to translating, which may be easier to integrate into the form factor of an existing transmission-based mammography unit. For the mammography system 800 shown in FIGS. 8A and 8B, there is an X-ray source 801, X-ray cone beam 802, collimator 803, breast compression paddles 804, tissue region of interest within patient breast 805, coded aperture 806, primary beam block 807, detector 808, component mounting/rotation system 809, display for acquired results 810, X-ray beam for X-ray scatter measurements 811, and scattered X-rays 812.

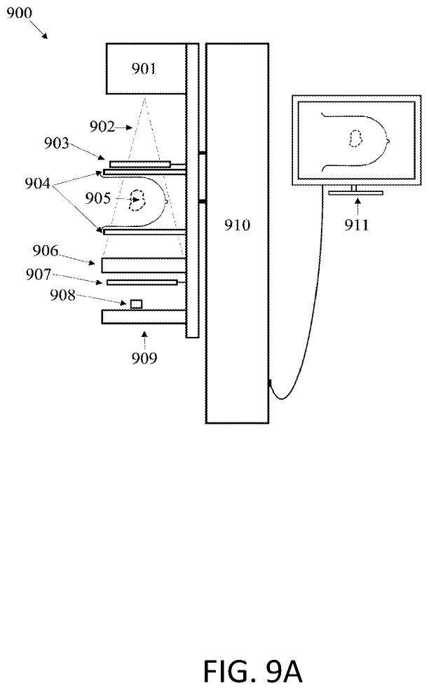

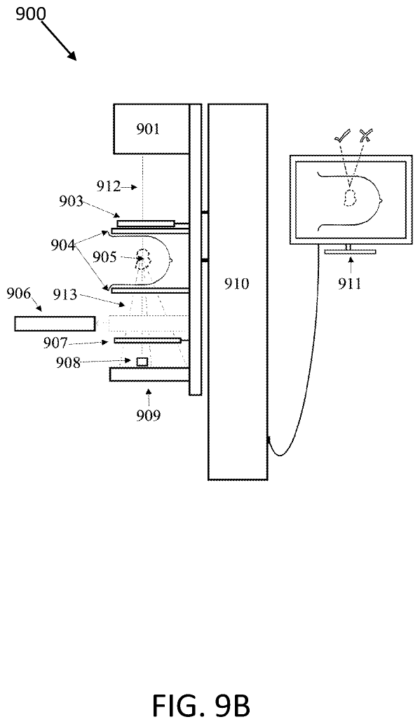

Another similar embodiment includes separate detectors for transmission and diffraction measurements and is shown in FIGS. 9A and 9B. For the mammography system 900 shown in FIGS. 9A and 9B, there is an X-ray source 901, X-ray cone beam 902, collimator 903, breast compression paddles 904, tissue region of interest within patient breast 905, X-ray transmission detector 906, coded aperture 907, primary beam block 908, X-ray scatter detector 909, component mounting/rotation system 910, display for acquired results 911, X-ray beam for X-ray scatter measurements 912, and scattered X-rays 913. In this case, once the transmission measurement is done, the transmission detector 906 translates or rotates out of the way to reveal the coded aperture 907, scatter detector 909, and beam block 908, while the components move to define the pencil or fan beam for the user selected region in the same way as in the embodiments of FIGS. 7A, 7B, 8A, and 8B.

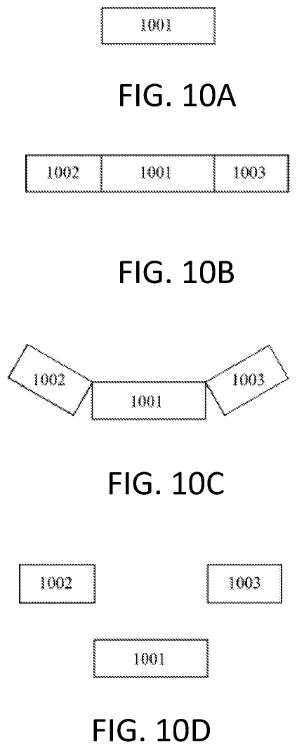

The embodiments of the mammography system that are described in the preceding paragraphs and shown in FIGS. 7A-9C are non-exhaustive examples of those that could be most easily implemented into the form factor of existing mammography systems. FIGS. 10A-10D demonstrate more specific X-ray detector arrangements for similar mammography systems within that class of embodiments, including arrangements with a single X-ray detector, multiple X-ray detectors, arrangements with multiple X-ray detectors at varying locations and/or varying orientations (effectively making a 3D arrangement of pixels), and arrangements with some measurement mode specific detectors, (e.g., multiple detectors, some of which are only for X-ray scatter measurements). In FIGS. 10A-10D, the primary X-ray detector 1001 can be used by itself or used in combination with any number of additional X-ray detectors (here 1002 and 1003) in various configurations.

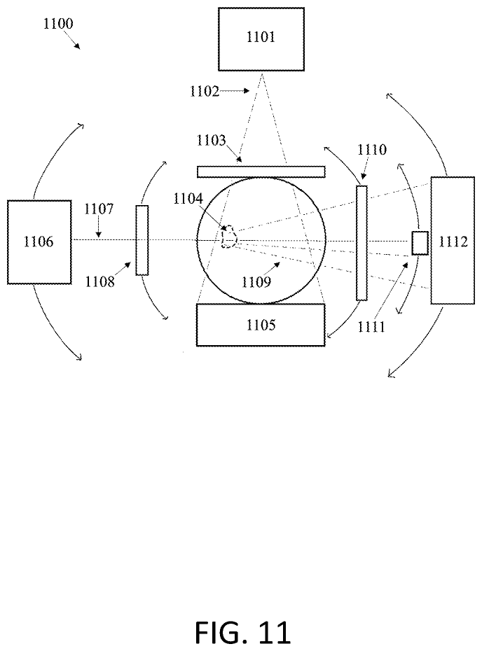

Another embodiment of the mammography system is shown in FIG. 11. For the mammography system 1100 shown in FIG. 11, there is a transmission X-ray source 1101, X-ray cone beam 1102, breast compression paddle 1103, tissue region of interest within patient breast 1104, X-ray transmission detector 1105, scatter X-ray source 1106, pencil/fan beam 1107, collimator 1108, X-ray scatter 1109, coded aperture 1110, primary beam block 1111, and X-ray scatter detector 1112. In this embodiment, there are two sources-one for performing the transmission measurement 1105, and one for performing the X-ray scatter measurement 1112. In this embodiment, the source used for the X-ray scatter measurement 1106, along with the accompanying collimator 1108, coded aperture 1110, beam block 1111, and detector 1112, can rotate to allow both for multiple perspectives of the same region of interest, which may be a user-specified subregion or the entire volume. An embodiment of the method that uses such a mammography system provides advantages such as allowing for optimization of the geometry for a given specified subregion, e.g. reducing path length from a suspicious mass to the exterior of the breast in the small angle forward scattering regime to reduce self-attenuation of the scatter signal in the breast tissue, minimizing dose to the patient, or improving the expected scatter reconstruction quality based on detector coverage or distance and relative orientation of expected scatter to coded aperture and detector.

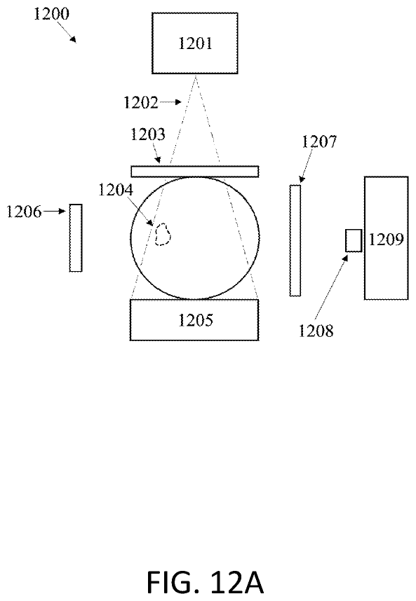

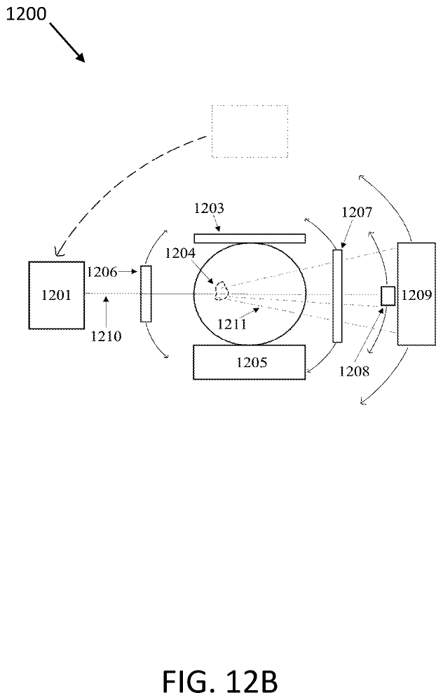

Another embodiment of the mammography system that allows for varying perspective on the breast tissue for the X-ray scatter measurement(s) is shown in FIGS. 12A and 12B. For the mammography system 1200 shown in FIGS. 12A and 12B, there is an X-ray source 1201, X-ray cone beam 1202, breast compression paddle 1203, tissue region of interest within patient breast 1204, X-ray transmission detector 1205, collimator 1206, coded aperture 1207, primary beam block 1208, X-ray scatter detector 1209, pencil/fan beam 1210, and X-ray scatter 1211. In this embodiment, instead of having a second X-ray source for the X-ray scatter measurement, the source used for the X-ray transmission measurement 1201 can rotate into a position to provide a significantly different perspective on the volume, effectively serving the purpose of the scatter source from the embodiment shown in FIG. 11. This embodiment is advantageous in reducing component and maintenance costs, while increasing reliability of the system by having fewer X-ray sources, which are typically the component with the shortest lifetime and the likeliest point of failure in an X-ray system.

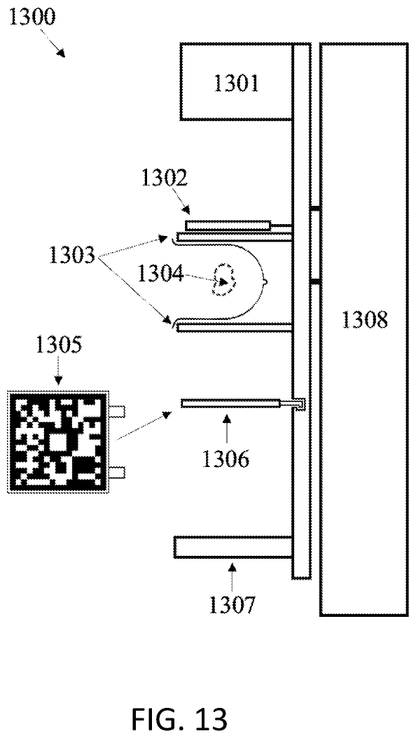

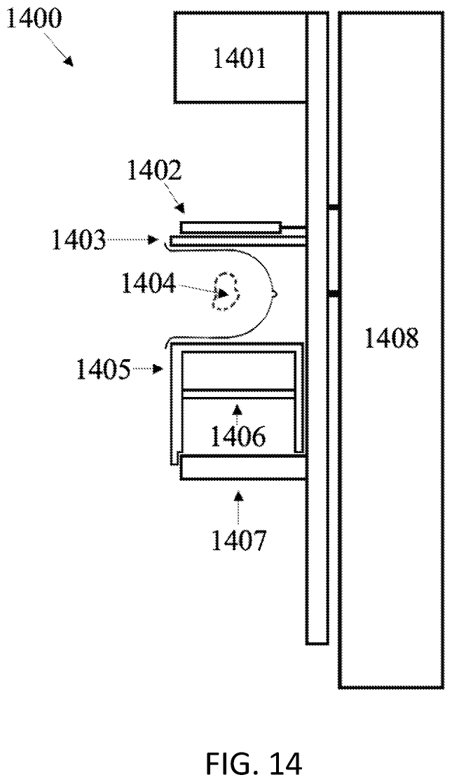

FIG. 13 depicts a mammography system with a detachable coded aperture similar to the mammography system depicted in FIG. 7. The coded aperture is attached between the breast of a patient and the X-ray detector for an X-ray scatter measurement. For the system 1300 shown in FIG. 13, there is an X-ray source 1301 with X-ray beams not shown, collimator 1302, breast compression paddles 1303, tissue region of interest within patient breast 1304, coded aperture 1305 with mechanism for attaching (e.g., clipping) to the device via device component 1306, X-ray detector 1307, and the body of the system 1308. Another similar embodiment includes combining the coded aperture into a magnification spacer table that rests on the detector. For the system 1400 shown in FIG. 14, there is an X-ray source 1401 with X-ray beams not shown, collimator 1402, top breast compression paddle 1403, tissue region of interest within patient breast 1404, magnification spacer table 1405 that rests or locks onto the X-ray detector, coded aperture 1406 that is combined with the magnification spacer table temporarily or permanently, X-ray detector 1407, and the body of the system 1408. This embodiment reflects an alternative method for magnification mode imaging, utilizing a raised platform that rests on the detector with a coded aperture attached or built into the platform between the top of the platform where the breast of a patient is positioned and the X-ray detector.

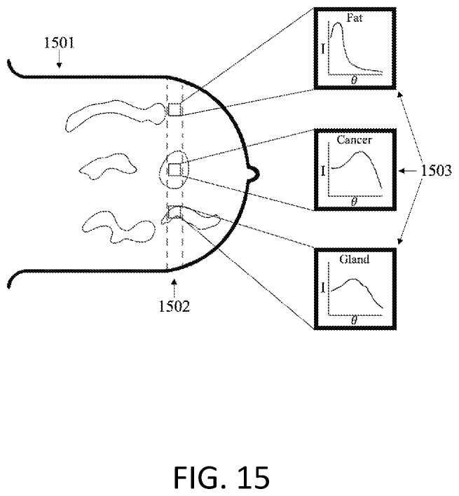

FIG. 15 presents an example representation of a portion of an X-ray scatter spectral reconstruction. Shown in FIG. 15 is a 2D or 3D X-ray radiodensity mammogram image produced from transmission data 1501, region where X-ray scatter measurements are taken 1502 marked between the dashed lines, and diffraction measurements that can be viewed for any tissue region of interest 1503 that can be used to differentiate fat, cancer, and glandular tissue. The spatially resolved reconstructed X-ray scatter spectra 1503 provides additional contrast for tissue differentiation. In typical operation of the system, such as by a technician or clinician, the reconstructed X-ray scatter spectra would not be accessible by the operator, but this data would be used to generate useful visualizations for the technician or clinician, such as a percentage match to reference spectra of specific tissue types, including healthy or cancerous tissue types, as shown in FIG. 3.

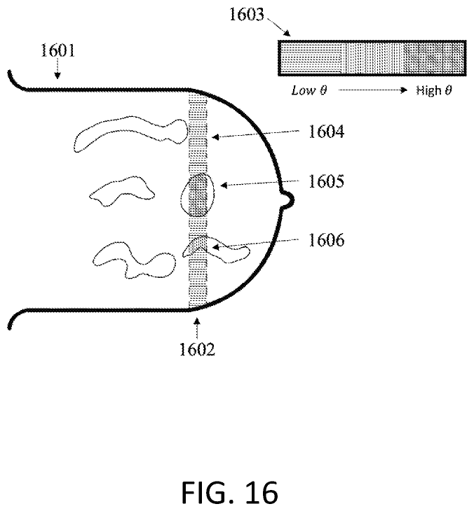

Another approach for using the X-ray scatter reconstruction to add clinically relevant visual contrast to a standard X-ray radiodensity mammogram generated from X-ray transmission measurements is shown in FIG. 16. Shown in FIG. 16 is a 2D or 3D standard mammogram image 1601, a region where X-ray scatter measurements are taken 1602 marked between the dashed lines, and color scale represented as different hatching patterns 1603. The color spectrum would be generated based on the X-ray scatter spectral reconstruction. A specific example would be transitioning between colors like blue, yellow, and red, for regions of X-ray scatter measurements with lower average scatter angles 1604, regions of X-ray scatter measurements with higher average scatter angles 1605, and regions of X-ray scatter measurements with middle average scatter angles 1606, respectively represented in FIG. 16 by only horizontal, both vertical and horizontal, and only vertical hatching patterns, respectively, where the determination of lower, higher, or medium average scatter angles correlate with the dominant feature or peak in reference spectra of specific healthy or cancerous tissue types. Other embodiments could utilize other calculated values from the x-ray scatter spectral reconstruction to generate colormaps that add additional contrast between tissue types.

The mammography system described herein further includes controllable electronics. In one embodiment, the mammography device includes components such as a processor, a system memory having a random-access memory (RAM) and a read-only memory (ROM), an I2C sensor, a system bus that couples the memory to the processor. The processor manages the overall operations of the mammography system. The processor is any controller, microcontroller, or microprocessor that is capable of processing program instructions. In one embodiment, the control electronics includes at least one antenna, which enables the mammography system to send information to at least one remote device and/or receive information from at least one remote device. The at least one antenna provides wireless communications, standards-based or non-standards-based including but not limited to, radiofrequency (RF), Wi-Fi, Bluetooth, Zigbee, near field communication (NFC), 3G, 4G, and/or 5G Cellular or other similar communication methods.

As further example, the processor may be a general-purpose microprocessor (e.g., a central processing (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, agate or transistor logic, discrete hardware components or any other suitable entity or combinations thereof that can perform calculations, process instructions for execution, and/or other manipulations of information.

The mammography system described herein is operable to analyze image data to determine whether to modify an X-ray beam and the positioning of the components of the mammography device. For example, and not limitation the at least one processor is designed to control the cross-sectional shape, divergence, and spatial extent of the initial X-ray beam(s) and one or more filters to modify the energy spectrum and irradiance of the X-ray beam(s) reaching the breast tissue, located between the X-ray source(s) and the breast tissue. Advantageously, if the at least one processor detects that the image data has poor quality, the at least one processor is operable to transmit a command to the x-ray source to change at least one of the shape, divergence, spatial extent, and/or intensity based on the quality.

Yet another advantage of the mammography system described herein is the ability to classify tissue based on image data (e.g., scatter data). Using a modulated coded aperture, the mammography system described herein is operable to receive image data corresponding to the breast of a patient and classify the breast tissue.

The mammography system described herein is further operable to analyze X-ray transmission data and determine a tissue type (e.g., adipose, normal, fibroglandular, cancer), optimize power supplied, system efficiency, a signal to noise ratio, an angle of scatter, momentum transfer. Advantageously, the mammography system described herein is operable to identify a healthy tissue margin based on the transmission image data and generate at least one recommendation based on the transmission image data. For example, and not limitation, the at least one recommendation includes identifying a region of interest to be removed. Another example includes a recommendation to modify and/or swap the coded aperture if the signal to noise ratio and/or received transmission data is insufficient.

In one embodiment, the mammography system described herein includes at least one algorithm designed to analyze the received image data to determine at least one tissue property (e.g., tissue classification). For example, and not limitation, the at least one algorithm includes a deblurring algorithm. In yet another embodiment, the at least one algorithm includes a machine learning algorithm. For example, and not limitation, the machine learning algorithm includes a supervised learning algorithm (e.g., classification), a semi-supervised learning algorithm, an unsupervised learning algorithm, and/or a reinforcement learning algorithm. In yet another example, the machine learning algorithm includes a Naïve Bayes algorithm, a K Means clustering algorithm, a Support Vector Machine (SVM) algorithm, a linear regression algorithm, a logistic regression algorithm, an artificial neural network, a decision trees, a random forest, a K-nearest-neighbor algorithm, a gradient boosting algorithm, and/or a dimensionality reduction algorithm.

There are several commonalities of the embodiments of the mammography system and method described above that provide key advantages when implemented. One such commonality is components that are used in both X-ray transmission measurements and X-ray scatter measurements. This allows for lower total system cost, less likelihood of component failure, and potential for decreased overall measurement time. Another commonality is the ability for key components (e.g., X-ray sources, collimators, coded apertures, and X-ray detectors) to rotate or translate. This allows for: certain combinations of the components to be used in one measurement mode (e.g., for X-ray scatter measurements or X-ray transmission measurements) and not the other, or both measurement modes, potentially saving on component costs; directing the X-ray source and accompanying X-ray scatter measurement components (e.g. pencil or fan beam collimators, coded apertures, beam blocks, detectors) in order to measure a specified sub-region of tissue, which could be used to lower patient dose or optimize measurements for reconstruction performance; measurements from multiple perspectives in either X-ray transmission or X-ray scatter modes, which could also be used to optimize performance; implementation of an embodiment of the system described herein that is readily integrated into an existing transmission-based mammography system.

Further specific aspects of potential embodiments of the general method may provide further advantages. One such specification is that the transmission and scatter measurements are performed synchronously over the same time period, which would reduce measurement time and could potentially reduce dose to the patient if the same beam was used synchronously for scatter and transmission measurements. The key components of the general system may vary in ways that would be common knowledge to those trained in the art and have obvious associated advantages in their implementation. These variations include but are not limited to variations in the type of X-ray source (e.g. X-ray generator versus radioactive isotope, generator anode material, generator focal spot size), variations in the type of X-ray detector (energy-integrating, stacked multi-energy channel, and energy discriminating or photon counting), variations in coded aperture pattern type (e.g. periodic, Fresnel zone plate, random or optimized random, uniformly redundant array), material, or thickness. Multiple coded apertures may also be used in combination or by themselves to modulate the detected scatter signal, the choice of which could be controlled automatically given the measurement conditions, e.g., given the geometric constraints of measuring scatter from a user-specified subregion of tissue. Existing mammography systems use digital pixelated area X-ray detectors to measure an X-ray transmission image of the breast of a patient. These include scintillator detectors such as those employing cesium iodide scintillators, or direct conversion detectors such as those employing amorphous selenium. While one of these commonly used energy-integrating X-ray transmission detectors would be sufficient for the present invention, detection of scattered X-rays by an energy differentiating or photon counting detector could provide for improved signal to noise ratio, dose reduction to patient, and overall performance of the system. There are also obvious advantages for scatter measurements to those skilled in the art for embodiments having a curved or staggered area configurations of pixels or detecting elements. FIGS. 9A, 9B, 11, 12A, and 12B show embodiments that use multiple detectors and could allow for these different types of X-ray detectors to be implemented, while FIG. 10 demonstrates example arrangements that multiple detectors (of potentially varying types) can be oriented in a general region for detecting transmitted and scattered X-rays.

In one embodiment, a spatially resolved volumetric X-ray scatter tomography mammography system is disclosed. The mammography system includes a movable X-ray source for generating a primary X-ray beam for irradiating a breast of a patient. The X-ray source is adjustable based on at least one operating parameter that includes exposure time, current, voltage, or filtration. The mammography system further includes a collimator positioned between the X-ray source and the breast of the patient. The collimator includes an opening that is configurable in at least one dimension to shape the primary X-ray beam. The mammography system further includes a plurality of movable breast plates operable to position the breast of the patient in a path of the primary X-ray beam. The mammography system further includes an X-ray detector array comprising a plurality of X-ray detecting elements. At least some of the X-ray detecting elements are movable. The X-ray detector array is configurable to position at least one X-ray detecting element distally from the X-ray source at a first measurement location in the path of the primary X-ray beam past the breast of the patient to measure transmitted X-ray radiation from the primary X-ray beam. The X-ray detector array is further configurable to position at least one X-ray detecting element distally from the X-ray source at a second measurement location outside the path of the primary X-ray beam past the breast of the patient to measure scattered X-ray radiation from the primary X-ray beam. The mammography system further includes a coded aperture positioned distally from the X-ray source between the breast of the patient and the X-ray detector array. The coded aperture is configured to modulate scattered X-ray radiation from the breast of the patient detected by the X-ray detector array. The mammography system is configurable to perform an X-ray scatter measurement and an X-ray transmission measurement. When performing the X-ray scatter measurement, the X-ray detector array is configured such that at least one X-ray detecting element is positioned out of the path of the primary X-ray beam to detect scattered X-ray radiation from the primary X-ray beam passing through the breast of the patient. When performing the X-ray transmission measurement, the X-ray detector array is configured such that at least one X-ray detecting element is positioned in the path of the primary X-ray beam to detect X-ray radiation transmitted through the breast of the patient. The mammography system further includes a control system comprising memory and a processor. The processor of the mammography system is configured for configuring the mammography system for the X-ray transmission measurement or the X-ray scatter measurement. Configuring the mammography system includes controlling at least one configuration parameter that includes a position of the X-ray source, a position of the X-ray detector array, a dimension of the opening of the collimator, at least one operating parameter of the X-ray source, or a position of at least one of the breast plates.

For the X-ray transmission measurement, the processor of the mammography system is further configured for receiving X-ray transmission data that represents transmitted X-ray radiation detected by the plurality of X-ray detecting elements of the X-ray detector array. The processor of the mammography system is further configured for producing an X-ray radiodensity mammogram image based on the received X-ray transmission data. The processor of the mammography system is further configured for identifying a region of interest in the breast of the patient based on the X-ray radiodensity mammogram image. The processor of the mammography system is further configured for determining at least one scatter configuration parameter for the X-ray scatter measurement based on the identified region of interest in the breast of the patient. The processor of the mammography system is further configured for configuring the mammography system for the X-ray scatter measurement based on the determined at least one scatter configuration parameter.

For the X-ray scatter measurement, the processor of the mammography system is further configured for receiving X-ray scatter data that represents scattered X-ray radiation for the region of interest detected by the plurality of X-ray detecting elements of the X-ray detector array. The processor of the mammography system is further configured for estimating a spatially resolved X-ray scatter spectral reconstruction based on the received X-ray scatter data, the received X-ray transmission data, and the at least one scatter configuration parameter. The processor of the mammography system is further configured for determining a spatially resolved tissue property based on the received X-ray scatter data of the region of interest. The processor of the mammography system is further configured for producing a spatially resolved scatter mammogram image based on the received X-ray transmission data and the received X-ray scatter data.

In one embodiment of the mammography system described herein, for the X-ray scatter measurement, the coded aperture is operable to move to a position distal from the X-ray source between the breast of the patient and the plurality of X-ray detecting elements to modulate the detected scattered X-ray radiation. For the X-ray transmission measurement, the breast of the patient is positioned at a first distance from the X-ray detector array. For the X-ray scatter measurement, the system may be further operable in a magnification mode. In the magnification mode, the breast of the patient is positioned at a second distance from the X-ray detector array. The second distance is greater than the first distance. The processor may be further configured determining a coded aperture configuration parameter that includes a position of the coded aperture during the X-ray scatter measurement of the identified region of interest in the breast of the patient. The processor may be further configured for controlling the position of the coded aperture based on the determined coded aperture configuration parameter.

In one embodiment of the mammography system described herein, the mammography system may further include a beam block configured to block the primary X-ray beam during the X-ray scatter measurement. The beam block is positioned between the breast of the patient and the X-ray detector array in the path of the primary X-ray beam. The beam block may be movable and controllable by the processor, and the processor may be further configured for determining a beam block configuration parameter that includes a position of the beam block during the X-ray scatter measurement of the identified region of interest in the breast of the patient. The processor may be further configured for controlling the position of the beam block based on the determined beam block configuration parameter.

In one embodiment of the mammography system described herein, the mammography system may further include an additional X-ray source. The X-ray source is configured for the X-ray transmission measurement and the additional X-ray source is configured for the scatter measurement.

In one embodiment of the mammography system described herein, the at least one spatially resolved tissue property includes a tissue type that indicates cancerous tissue or benign tissue. The spatially resolved scatter mammogram image includes at least one indication based on the spatially resolved X-ray scatter spectral reconstruction. The at least one indication indicates whether the region of interest includes benign tissue or cancerous tissue.

In one embodiment of the mammography system described herein, the processor may be further configured for generating a colorization of the spatially resolved scatter mammogram image produced based on the received X-ray transmission data and the received X-ray scatter data. The colorization is based on the spatially resolved X-ray scatter spectral reconstruction.

In one embodiment of the mammography system described herein, the processor may be further configured for calculating a spatially resolved estimate of momentum transfer spectra of the breast of region of interest from the received X-ray scatter data. The processor may be further configured for using the spatially resolved estimate of the momentum transfer spectra in the determination of the spatially resolved tissue property. The memory may include a reference library of a plurality of existing tissue momentum transfer spectra. The processor may be further configured for using the reference library of existing tissue momentum transfer spectra in combination with the calculated spatially resolved estimate of the momentum transfer spectra of region of interest to determine the spatially resolved tissue property. The processor may be further configured for using a classification algorithm in the determination of the spatially resolved tissue property. The processor may be further configured for using a machine-learning algorithm in the calculation of the spatially resolved estimate of the spatially resolved tissue property. The processor may be further configured for using a rules-based classification algorithm in the calculation of the spatially resolved estimate of the spatially resolved tissue property.

In one embodiment of the mammography system described herein, at least one of the X-ray detecting elements is used for both the X-ray transmission measurement and the X-ray scatter measurement.

In one embodiment of the mammography system described herein, the processor is further configured for changing the position of the X-ray source to control an angle of incidence of the primary X-ray beam relative to the breast of the patient.

In one embodiment of the mammography system described herein, the at least one operating parameter of the X-ray source is configurable to control an irradiance and an energy spectrum of the primary X-ray beam.

In one embodiment of the mammography system described herein, for the X-ray scatter measurement, at least one of the X-ray detecting elements is configurable for detecting X-ray radiation from the primary X-ray beam transmitted directly through the breast of the patient. The processor may be further configured for receiving X-ray transmission data detected by the X-ray detecting elements that are configured for detecting X-ray radiation from the primary X-ray beam transmitted directly through the breast of the patient. The received X-ray transmission data represents the detected transmitted X-ray radiation during the X-ray scatter measurement. The estimation of the spatially resolved X-ray scatter spectral reconstruction is further based on the received X-ray transmission data detected during the X-ray scatter measurement.

In one embodiment of the mammography system described herein, the processor is further configured for estimating a dose of radiation of the patient during the X-ray scatter measurement and determining the at least one scatter configuration parameter for the X-ray scatter measurement based on the estimated dose of radiation to the patient.

In an embodiment, a method of performing spatially resolved volumetric X-ray diffraction tomography mammography is disclosed. The method includes performing an X-ray transmission measurement. The X-ray transmission measurement is performed by transmitting a first primary X-ray beam from an X-ray source through a collimator having an opening configurable in at least one dimension to shape the first primary X-ray beam and through a breast of a patient positioned between a plurality of breast plates. The X-ray source is adjustable based on at least one operating parameter that includes exposure time, current, voltage, and/or filtration. The X-ray transmission measurement is further performed by detecting X-ray radiation from the first primary X-ray beam transmitted directly through the breast of the patient using a plurality of X-ray detecting elements of an X-ray detector array. At least some of the X-ray detecting elements are movable. The X-ray transmission measurement is further performed by receiving X-ray transmission data via a control system including a processor and memory from the plurality of X-ray detecting elements. The X-ray transmission data represents the detected X-ray radiation from the first primary X-ray beam after the first primary X-ray beam has passed through the collimator and the breast of the patient. The X-ray transmission measurement is further performed by producing a radiodensity mammogram image of the breast of the patient based on the received X-ray transmission data. For example, and not limitation, the radiodensity mammogram image is two-dimensional or three-dimensional. The method further includes identifying a region of interest in the breast of the patient based on the radiodensity mammogram image. The method further includes determining a scatter measurement configuration parameter for an X-ray scatter measurement of the identified region of interest in the breast of the patient. The scatter measurement configuration parameter includes a position of the X-ray source, a position of the plurality of the X-ray detecting elements, a dimension of the opening of the collimator, an X-ray source operating parameter, and/or a position of at least one of the plurality of breast plates. The method further includes performing the X-ray scatter measurement of the region of interest. The X-ray scatter measurement of the region of interest is performed by transmitting a second primary X-ray beam from the X-ray source through the collimator to shape the second primary X-ray beam and through the breast of the patient positioned with the plurality of breast plates. The X-ray scatter measurement of the region of interest is further performed by modulating scattered X-ray radiation from the second primary X-ray beam interacting with the breast of the patient using a coded aperture positioned between the breast of the patient and the plurality of X-ray detecting elements. The X-ray scatter measurement of the region of interest is further performed by detecting the modulated scattered X-ray radiation using the plurality of X-ray detecting elements. The X-ray scatter measurement of the region of interest is further performed by receiving X-ray scatter data representing detected scattered X-ray radiation from the plurality of X-ray detecting elements. The X-ray scatter measurement of the region of interest is further performed by calculating a spatially resolved X-ray scatter spectral reconstruction based on the received X-ray scatter data, the received X-ray transmission data, and the determined scatter measurement configuration parameter. The X-ray scatter measurement of the region of interest is further performed by determining at least one spatially resolved tissue property based on the received X-ray scatter data of the region of interest. The X-ray scatter measurement of the region of interest is further performed by producing a spatially resolved scatter mammogram image based on the received X-ray transmission data and the received X-ray scatter data.

In one embodiment of the method of performing spatially resolved volumetric X-ray diffraction tomography mammography, the coded aperture is moved to a position distal from the X-ray source between the breast of the patient and the plurality of X-ray detecting elements to modulate the detected scattered X-ray radiation from the second primary X-ray beam during the X-ray scatter measurement. When performing the X-ray transmission measurement, the breast of the patient is positioned at a first distance from the plurality of the X-ray detecting elements, and when performing the X-ray scatter measurement, a magnification mode is used in which the breast of the patient is positioned at a second distance from the plurality of the X-ray detecting elements. The second distance is greater than the first distance. The method may further include determining, via the processor, a configuration parameter that represents the position of the coded aperture during the X-ray scatter measurement of the identified region of interest in the breast of the patient and controlling, via the processor, the position of the coded aperture.

In one embodiment of the method of performing spatially resolved volumetric X-ray diffraction tomography mammography, the method may further include blocking the second primary X-ray during the X-ray scatter measurement with a beam block positioned in the path of the second primary X-ray beam between the breast of the patient and the plurality of X-ray detecting elements. The beam block is movable, and when performing the X-ray scatter measurement, the beam block is positioned between the breast of the patient and the X-ray detecting elements in the path of the second primary X-ray beam. The method may further include determining, via the processor, a beam block configuration parameter that represents the position of the beam block during the X-ray scatter measurement of the identified region of interest and controlling, via the processor, the position of the beam block based on the determined beam block configuration parameter.

In one embodiment of the method of performing spatially resolved volumetric X-ray diffraction tomography mammography, the X-ray source is used for the X-ray transmission measurement and an additional X-ray source is used for the X-ray scatter measurement.

In one embodiment of the method of performing spatially resolved volumetric X-ray diffraction tomography mammography, the at least one spatially resolved tissue property includes a tissue type that indicates cancerous tissue or benign tissue. The spatially resolved scatter mammogram image includes at least one indication based on the spatially resolved X-ray scatter spectral reconstruction. The at least one indication indicates whether the identified region of interest includes benign tissue or cancerous tissue.

In one embodiment of the method of performing spatially resolved volumetric X-ray diffraction tomography mammography, the method further includes generating a colorization of the spatially resolved scatter mammogram image based on the received X-ray transmission data and the received X-ray scatter data. The colorization is determined based on the spatially resolved X-ray scatter spectral reconstruction.

In one embodiment of the method of performing spatially resolved volumetric X-ray diffraction tomography mammography, the method further includes calculating a spatially resolved estimate of momentum transfer spectra of the region of interest from the received X-ray scatter data. The method further includes using the spatially resolved estimate of the momentum transfer spectra in the determination of the at least one spatially resolved tissue property. The method may further include using a reference library of a plurality of existing tissue momentum transfer spectra in combination with the calculated spatially resolved estimate of the momentum transfer spectra of the region of interest to determine the at least one spatially resolved tissue property. The method may further include using a classification algorithm when determining the at least one spatially resolved tissue property. The method may further include using a machine-learning algorithm in the calculation of the spatially resolved estimate of the at least one spatially resolved tissue property. The method may further include using a rules-based classification algorithm in the calculation of the spatially resolved estimate of the at least one spatially resolved tissue property.

In one embodiment of the method of performing spatially resolved volumetric X-ray diffraction tomography mammography, the method further includes using at least one X-ray detecting element for both the X-ray transmission measurement and the X-ray scatter measurement.

In one embodiment of the method of performing spatially resolved volumetric X-ray diffraction tomography mammography, the method further includes controlling the position of the X-ray source to control an angle of incidence of the first primary X-ray beam or the second primary X-ray beam relative to the breast of the patient.

In one embodiment of the method of performing spatially resolved volumetric X-ray diffraction tomography mammography, the method further includes configuring at least one operating parameter of the X-ray source to control an irradiance and an energy spectrum of the first primary X-ray beam or the second primary X-ray beam.

In one embodiment of the method of performing spatially resolved volumetric X-ray diffraction tomography mammography, the method further includes, for the X-ray scatter measurement, receiving X-ray transmission data detected by the X-ray detecting elements that are configured for detecting X-ray radiation from the second primary X-ray beam transmitted directly through the breast of the patient. The received X-ray transmission data represents the detected transmitted X-ray radiation during the X-ray scatter measurement. The calculation of the spatially resolved X-ray scatter spectral reconstruction is further based on the received X-ray transmission data detected during the X-ray scatter measurement. At least one X-ray detecting element is configurable for detecting X-ray radiation from the second primary X-ray beam transmitted directly through the breast of the patient.

In one embodiment of the method of performing spatially resolved volumetric X-ray diffraction tomography mammography, the method further includes, for the X-ray scatter measurement, estimating a dose of radiation to the patient, and determining the at least one scatter configuration parameter for the X-ray scatter measurement based on the estimated dose of radiation to the patient.

Any combination of one or more computer-readable medium(s) may be utilized with the mammography system described herein. The computer-readable medium may be a computer readable signal medium or a computer-readable storage medium (including, but not limited to, non-transitory computer-readable storage media). A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer readable storage medium and that can communicate, propagate, or transport a program for use by or in connection with an instruction execution system, apparatus, or device.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications, or other data embodying any one or more of the methodologies or functions described herein. The computer readable medium may include the memory, the processor, and/or the storage media and may be a single medium or multiple media (e.g., a centralized or distributed computer system) that stores the one or more sets of instructions. Non-transitory computer readable media includes all computer readable media, with the sole exception being a transitory, propagating signal per se. The instructions may further be transmitted or received over the network via the network interface unit as communication media, which may include a modulated data signal such as a carrier wave or other transport mechanism and includes any delivery media. The term “modulated data signal” means a signal that has one or more of its characteristics changed or set in a manner as to encode information in the signal.

The corresponding structures, materials, acts, and equivalents of all means or step plus function elements in the claims below are intended to include any structure, material, or act for performing the function in combination with other claimed elements as specifically claimed. The description of the mammography system described herein has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the mammography system described herein have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A spatially resolved volumetric X-ray scatter tomography mammography system, the mammography system comprising:
an X-ray source for generating a primary X-ray beam for irradiating a breast of a patient;
a collimator positioned between the X-ray source and the breast of the patient;
a plurality of movable breast plates operable to position the breast of the patient in a path of the primary X-ray beam;
an X-ray detector array comprising a plurality of X-ray detecting elements, at least some of which are movable, wherein the X-ray detector array is configurable to:
position at least one X-ray detecting element distally from the X-ray source at a first measurement location in the path of the primary X-ray beam past the breast of the patient to measure transmitted X-ray radiation from the primary X-ray beam, and
position at least one X-ray detecting element distally from the X-ray source at a second measurement location outside the path of the primary X-ray beam past the breast of the patient to measure scattered X-ray radiation from the primary X-ray beam;
a coded aperture positioned distally from the X-ray source between the breast of the patient and the X-ray detector array, wherein the coded aperture is configured to modulate scattered X-ray radiation from the breast of the patient detected by the X-ray detector array;
a control system comprising a memory and a processor, wherein the processor is configured to:
control at least one configuration parameter that includes a position of the X-ray source, a position of the X-ray detector array, a dimension of an opening of the collimator, at least one operating parameter of the X-ray source, or a position of at least one of the breast plates;
produce an X-ray radiodensity mammogram image based on a received X-ray transmission data;
identify a region of interest in the breast of the patient based on the X-ray radiodensity mammogram image;
determine at least one scatter configuration parameter for the X-ray scatter measurement based on the identified region of interest in the breast of the patient; and
configure the mammography system for the X-ray scatter measurement based on the determined at least one scatter configuration parameter;
receive X-ray scatter data that represents scattered X-ray radiation for the region of interest detected by the plurality of X-ray detecting elements of the X-ray detector array;
estimate a spatially resolved X-ray scatter spectral reconstruction based on the received X-ray scatter data, the received X-ray transmission data, and the at least one scatter configuration parameter; and
determine a spatially resolved tissue property based on the received X-ray scatter data of the region of interest;
wherein the mammography system is configurable to perform an X-ray scatter measurement and an X-ray transmission measurement.

2. The mammography system of claim 1, wherein, for the X-ray scatter measurement, the coded aperture is operable to move to a position distal from the X-ray source between the breast of the patient and the plurality of X-ray detecting elements to modulate the detected scattered X-ray radiation.

3. The mammography system of claim 2, wherein, for the X-ray transmission measurement, the breast of the patient is positioned at a first distance from the X-ray detector array, and for the X-ray scatter measurement, the mammography system is further operable in a magnification mode, wherein, in the magnification mode, the breast of the patient is positioned at a second distance from the X-ray detector array, wherein the second distance is greater than the first distance.

4. The mammography system of claim 2, wherein the processor is further configured to:
determine a coded aperture configuration parameter that includes a position of the coded aperture during the X-ray scatter measurement of the identified region of interest in the breast of the patient, and
control the position of the coded aperture based on the determined coded aperture configuration parameter.

5. The mammography system of claim 1, further comprising a beam block configured to block the primary X-ray beam during the X-ray scatter measurement, wherein the beam block is positioned between the breast of the patient and the X-ray detector array in the path of the primary X-ray beam.

6. The mammography system of claim 5, wherein the beam block is movable and controllable by the processor, and wherein the processor is further configured for:
determining a beam block configuration parameter that includes a position of the beam block during the X-ray scatter measurement of the identified region of interest in the breast of the patient, and
controlling the position of the beam block based on the determined beam block configuration parameter.

7. The mammography system of claim 1, further comprising a second X-ray source, wherein the X-ray source is configured for the X-ray transmission measurement and the second X-ray source is configured for the X-ray scatter measurement.

8. The mammography system of claim 1, wherein the spatially resolved tissue property comprises a tissue type that indicates cancerous tissue or benign tissue.

9. (canceled)

10. (canceled)

11. The mammography system of claim 1, wherein the processor is further configured to:
calculate a spatially resolved estimate of momentum transfer spectra of the region of interest from the received X-ray scatter data; and
determine spatially resolved tissue property using the spatially resolved estimate of the momentum transfer spectra of the region of interest.

12. The mammography system of claim 11, wherein:
the processor is configured to determine the spatially resolved tissue property using a reference library of a plurality of existing tissue momentum transfer spectra in combination with the spatially resolved estimate of the momentum transfer spectra of the region of interest, wherein the reference library of a plurality of existing tissue momentum transfer spectra are stored on the memory.

13. The mammography system of claim 11, wherein the processor is further configured to determine the spatially resolved tissue property using a classification algorithm, wherein the classification algorithm is a machine-learning algorithm or a rules-based classification algorithm.

14. (canceled)

15. (canceled)

16. The mammography system of claim 1, wherein at least one of the X-ray detecting elements is used for both the X-ray transmission measurement and the X-ray scatter measurement.

17. The mammography system of claim 1, wherein the processor is further configured to
change the position of the X-ray source to control an angle of incidence of the primary X-ray beam relative to the breast of the patient.

18. The mammography system of claim 1, wherein the at least one operating parameter of the X-ray source is configurable to control an irradiance and an energy spectrum of the primary X-ray beam.

19. The mammography system of claim 1, wherein, for the X-ray scatter measurement:
at least one of the X-ray detecting elements is configurable for detecting X-ray radiation from the primary X-ray beam transmitted directly through the breast of the patient; and
the processor is further configured for receiving X-ray transmission data detected by the X-ray detecting elements that are configured for detecting X-ray radiation from the primary X-ray beam transmitted directly through the breast of the patient, wherein the received X-ray transmission data represents the detected transmitted X-ray radiation during the X-ray scatter measurement;
wherein the estimation of the spatially resolved X-ray scatter spectral reconstruction is further based on the received X-ray transmission data detected during the X-ray scatter measurement.

20. The mammography system of claim 1, wherein the processor is further configured for:
estimating a dose of radiation to the patient during the X-ray scatter measurement; and
determining the at least one scatter configuration parameter for the X-ray scatter measurement based on the estimated dose of radiation to the patient.

21-62. (canceled)

63. The mammography system of claim 1, wherein the processor is further configured to produce a spatially resolved scatter mammogram image based on the received X-ray transmission data and the received X-ray scatter data.

64. The mammography system of claim 63, wherein the spatially resolved scatter mammogram image comprises at least one indication based on the spatially resolved X-ray scatter spectral reconstruction, wherein the at least one indication indicates the determined spatially resolved tissue property of the region of interest.

65. The mammography system of claim 63, wherein the processor is further configured to generate a colorization of the spatially resolved scatter mammogram image produced based on the received X-ray transmission data and the received X-ray scatter data, wherein the colorization is based on the spatially resolved X-ray scatter spectral reconstruction.

66. A method of performing spatially resolved volumetric X-ray scatter tomography mammography, the method comprising:
performing an X-ray transmission measurement; and producing a radiodensity mammogram image of a breast of a patient based on a received X-ray transmission data;
identifying a region of interest in the breast of the patient based on the radiodensity mammogram image;
determining a scatter measurement configuration parameter for an X-ray scatter measurement of the identified region of interest, wherein the scatter measurement configuration parameter comprises a position of an X-ray source, a position of a plurality of X-ray detecting elements, a dimension of an opening of a collimator, an X-ray source operating parameter, or a position of at least one of a plurality of breast plates;
performing the X-ray scatter measurement of the region of interest by:
transmitting a second primary X-ray beam from the X-ray source through the collimator to shape the second primary X-ray beam and through the breast of the patient positioned with the plurality of breast plates;
modulating scattered X-ray radiation from the second primary X-ray beam interacting with the breast of the patient using a coded aperture positioned between the breast of the patient and the plurality of X-ray detecting elements;
detecting the modulated scattered X-ray radiation using the plurality of X-ray detecting elements;
receiving X-ray scatter data representing detected scattered X-ray radiation from the plurality of X-ray detecting elements;
calculating a spatially resolved X-ray scatter spectral reconstruction based on the received X-ray scatter data, the received X-ray transmission data, and the determined scatter measurement configuration parameter;
determining at least one spatially resolved tissue property based on the received X-ray scatter data of the region of interest.
